(19) United States
(12) Patent Application Publication
(10) Pub. No.: US 2012/0064180 A1
(43) Pub. Date: Mar. 15, 2012

(54) USE OF NO AND NO DONORS FOR TERMINATING DORMANCY IN INVERTEBRATES

(75) Inventors: Peter Bossier, De Pinte (BE); Guy Smagghe, Temse (BE)

(73) Assignee: UNIVERSITEIT GENT, Gent (BE)

(21) Appl. No.: 13/320,300

(22) PCT Filed: May 12, 2010
(86) PCT No.: PCT/EP2010/056526

§ 371 Date: Nov. 14, 2011

(30) Foreign Application Priority Data
May 15, 2009 (GB) 0908289.2

(51) Int. Cl. A61K 33/00; A61K 31/15

(52) U.S. Cl. 424/718; 514/231.2; 514/611

(57) ABSTRACT

The present invention relates to a method for terminating dormancy in an invertebrate, wherein said invertebrate is brought in contact with nitric oxide and/or a nitric oxide donor. The method can be used in pest management of unfavourable species, in eliminating dormant stages of aquatic invertebrates in ballast water in promoting termination of dormancy in beneficial species and/or in stimulating production of live food in aquacultures.

The present invention relates to a method for terminating dormancy in an invertebrate, wherein said invertebrate is brought in contact with nitric oxide and/or a nitric oxide donor. The method can be used for promoting termination of dormancy in beneficial invertebrates but also for eliminating unfavourable terrestrial and aquatic species in pest management.

### BACKGROUND OF THE INVENTION

Nitric oxide (NO) is a bioactive molecule that exerts a number of diverse activities in phylogenetically distant species, as well as opposing effects in related biological systems. It was firstly described in mammals as a major messenger in the cardiovascular, immune and nervous system, in which it plays regulatory, signaling, cytoprotective and cytotoxic effects. This versatility is mainly achieved through interactions with targets via either a redox or an additive chemistry. For this reason, metal- and thiol-containing proteins serve as major target sites for NO: these include signaling proteins, receptors, enzymes, transcription factors and DNA, among others. Furthermore, NO is a small, highly diffusible molecule. It rapidly crosses biological membranes and triggers various different processes in a short period of time. In this context, NO can co-ordinate and regulate cellular functions of microsomes and organelles such as mitochondria.

Only few data are available about the role of NO in invertebrates. Hodin J. (2006) has reported the repression of NO during metamorphic events linked to a shift in habitat function.

In particular, the use of NO/cyclic-guanosine monophosphate (cGMP) signalling as a repressor of settlement appears to be a common feature in sea urchins (Echinodermata), sea squirts (Chordata: Tunicata), and a gastropod (Mollusca) (Bishop C D et al., 2003). Settlement is the metamorphic event wherein a free-swimming planktonic form becomes a benthic organism. As hypothesized by Bishop, et al. (2006) NO “repression” of settlement may be selectively advantageous in organisms that use a wide range of possible settlement inducers as a way of preventing accidental, precocious, or otherwise inappropriate settlement. These data suggest that the utility of NO as a repressor of settlement depends on the precise ecological requirements of the settling larva.

Holometabolous insects represent a parallel case. There seems to be a direct, antagonistic interaction of ecdysteroids and NO production during insect metamorphosis (Gammie S C et al., 1999). On the other hand, ecdysteroids seem to regulate adult eye morphogenesis in the tobacco hornworm Manduca sexta via a nitric oxide signaling pathway (Champlin et al. 2000).

A putative role of NO in invertebrates, contrary to its role as a repressor of settlement, has also been reported under stress conditions like oxidative stress. Drosophila embryos rapidly arrest development upon severe hypoxia and recover upon restoration of oxygen, even days later. NO, a putative hypoxia signal, induced a reversible arrest of development, gene expression and turnover. Reciprocally, a NO scavenger allowed continued gene expression and turnover during hypoxia, but it reduced hypoxia tolerance. Teodoro and O'Farrell (2003) suggested that hypoxia-induced stasis preserves the status quo of embryonic processes and promotes survival and that nitric oxide is a mediator of this response. In the dormant stages of the snail Helix lucorum on the other hand NO synthesis seemed to be blocked (Röszer T et al., 2004).

Different from higher organisms like mammals, throughout their life cycle invertebrates will have to go through different phases with different metabolic activity. Metamorphosis for example, usually proceeds in distinct stages, starting with larva or nymph, optionally passing through pupa, and ending as adult.

In hemimetabolism, the development of larva often proceeds in repeated stages of growth and ecdysis (moulting), these stages are called instars. The juvenile forms closely resemble adults, but are smaller and, if the adult has wings, lack wings.

Invertebrates may undergo stages of higher and lower metabolic activity. For example, in response of unfavorable environmental conditions invertebrates may enter into a dormant stage. Dormancy, more in particular diapause, is a dynamic process consisting of several distinct phases. While dormancy varies considerably from one taxon of organism to another, these phases can be characterized by particular sets of metabolic processes and responsiveness of the organism to certain environmental stimuli. The induction phase occurs at a genetically predetermined stage of life and occurs well in advance of the environmental stress. This sensitive stage may occur within the lifetime of the diapausing individual, or in proceeding generations, particularly in egg dormancy. During this phase, the organism is responsive to external cues called token stimuli, which trigger the switch from direct development pathways to dormancy pathways. Token stimuli can consist of changes in photoperiod, thermoperiod or allochemicals. These stimuli are not in themselves favourable or unfavourable to development, but they herald an impending change in environmental conditions. The preparation phase usually follows the induction phase, though the individual may go directly from induction to initiation without a preparation phase. During this phase, subjects accumulate and store molecules such as lipids, proteins and carbohydrates. These molecules are used to maintain the organism throughout dormancy and to provide fuel for development following dormancy termination. In insects the composition of the cuticle may be altered by changing hydrocarbon composition and by adding lipids to reduce water loss. The initiation phase begins when morphological development ceases. In some cases, this change may be very distinct and can involve moulting into a specific dormancy stage, or be accompanied by colour change. Enzymatic changes may take place in preparation for cold hardening. The organism may also undergo behavioural changes and begin to aggregate, migrate or search for suitable overwintering sites. During the maintenance phase, subjects experience lowered metabolism and developmental arrest is maintained. Sensitivity to certain stimuli which act to prevent termination of dormancy, such as photoperiod and temperature, is increased. At this stage, organisms are unresponsive to changes in the environment that will eventually trigger the end of dormancy, but they grow more sensitive to these stimuli as time progresses. In organisms that undergo obligate dormancy, termination may occur spontaneously, without any external stimuli. In the relatively few species in which a specific factor is needed to terminate autumnal-hibernal dormancy, four stimuli have been identified: photoperiod, food, moisture, and an internal stimulus from insect host or parasite. In facultative dormancy, token stimuli must occur to terminate dormancy. These stimuli may include chilling, freezing or contact with water, depending on the environmental conditions being avoided. These stimuli are important in preventing the subject from terminating dormancy too soon, for instance in response to warm weather in late fall. Termination may occur at the height of unfavourable conditions, such as in the middle of winter. Over time, depth of dormancy slowly decreases until direct development can resume, if conditions are favourable. Dormancy frequently ends prior to the end of unfavourable conditions and is followed by a state of post-dormancy quiescence from which the insect can arouse and begin direct development, should conditions change to become more favourable. This allows the insect to continue to withstand harsh conditions while being ready to take advantage of good conditions as rapidly as possible.

Dormancy in the tropics is often in response to biotic rather than abiotic factors. For example, food in the form of vertebrate carcasses may be more abundant following dry seasons, or oviposition sites in the form of fallen trees may be more available following rainy seasons. Also, dormancy may serve to synchronize mating seasons or reduce competition, rather than to avoid unfavourable climatic conditions.

As is evident from the foregoing, it is of utmost importance for the survival of invertebrates that the different life stages, including dormancy, metamorphosis and growth are closely regulated at several levels. Environmental stimuli interact with genetic pre-programming to affect metabolic and enzymatic changes and from the above, NO may have a distinct and specific role in either of said phases.

Environmental regulators of dormancy generally display a characteristic seasonal pattern. In temperate regions, photoperiod is one of the most reliable cues of seasonal change. Depending on the season in which dormancy occurs, either short or long days can act as token stimuli. Subjects may also respond to changing day length as well as relative day length. Temperature may also act as a regulating factor, either by inducing dormancy or, more commonly, by modifying the response of the individual to photoperiod. Invertebrates may respond to thermoperiod, the daily fluctuations of warm and cold that correspond with day and night, as well as to absolute or cumulative temperature. Food availability and quality may also help regulate dormancy. After dormancy terminates, some species require a specific stimulus, such as water absorption, to initiate development. However, the majority of species depend on the occurrence of temperatures within the limits set by the upper and lower thermal thresholds for growth. Physical factors during dormancy can profoundly influence postdormancy development and reproduction.

It has previously been demonstrated that NO signalling is repressed during settlement, metamorphosis and dormancy and that NO induction promotes stasis and survival during hypoxia. Unexpectedly it has now been found that NO and NO donors terminate dormancy in invertebrates. As will be explained in more detail in the examples hereinafter, NO and NO donors can be utilized to terminate dormant stages of invertebrates, hence NO and NO donors can be used in pest management of unfavourable species, to eliminate dormant stages of aquatic invertebrates in ballast water to promote termination of dormancy in beneficial species and/or to stimulate production of live food in aquacultures.

### SUMMARY OF THE INVENTION

The present invention relates to a method for terminating dormancy in an invertebrate characterized in that said dormant invertebrate is brought in contact with nitric oxide and/or a nitric oxide donor. Said dormant invertebrate can be in an embryonic or a post-embryonic stage and/or can be an aquatic or terrestrial invertebrate. In particular said dormant invertebrate can be from the Insecta, Crustaceae, Nematoda or Rotifera.

In one aspect, the method of the invention is performed under predetermined conditions. Said predetermined conditions can be selected from the group of photoperiod, temperature, food, moisture, salt, oxygen, chemicals or endocrine factors.

In another aspect of the method of the invention the nitric oxide donor is selected from Spermine NONOate, Papa NONOnate, Sin-1 Chloride, S-nitrosoglutathione, S-nitrosocysteine, sodium nitroprusside, S-Nitroso-N-penicillamine (SNAP), Glyco-SNAP-1, Glyco-SNAP-2, Diethylamine NONOate, Diethylamine NONOate/AM, NOC-5, NOC-7, NOC-9, NOC-12, NOC-18, NOR-1, or NOR-3. The nitric oxide can also be delivered by microorganisms, in particular bacteria.

In a further aspect of the invention termination of dormancy can be determined using one or more of a change in an embryonic stage, a post embryonic stage, an endocrine factor, an expression of a gene, an expression of a protein, a signalling process, the state of the reproductive system, an enzyme activity, a metabolic activity, the composition of the cuticle, the respiration rate, and the behavior more in particular the feeding behavior.

The present invention is also concerned with a kit comprising nitric oxide, a nitric oxide donor or nitric oxide delivering microorganisms, and a dormant invertebrate. It is further concerned with a container wherein said nitric oxide, nitric oxide donor or nitric oxide delivering microorganisms are mixed with and/or adhered or coated onto said dormant invertebrate.

Another aspect of the invention relates to the use of nitric oxide or a nitric oxide donor for terminating dormancy in an invertebrate and to study termination of dormancy. Said dormant invertebrate can be beneficial or unfavorable. Termination of dormancy in beneficial invertebrates can be used to produce live food, in particular for aquacultures. Termination of dormancy in unfavorable invertebrates can be used to eliminate unfavorable invertebrates, in pest control management. Termination of dormancy can subsequently be followed by treatment with a control agent. Termination of dormancy can be determined using one or more of a change in an embryonic stage, a post embryonic stage, an endocrine factor, an expression of a gene, an expression of a protein, a signaling process, the state of the reproductive system, an enzyme activity, a metabolic activity, the composition of the cuticle, the respiration rate, and the behavior more in particular the feeding behavior.

### BRIEF DESCRIPTION OF THE FIGURES

FIG. 1. Artemia life history stages used for developmental quantification. Artemia were fixed in Lugol's solution and photographed with a Nikon AZ 100 microscope. A, an E1 larva emerging through the crack in a cyst shell; B, a completely emerged E2 larva enclosed in a hatching membrane and attached to a cyst shell; C, an E3 larva released from a cyst but enclosed in a hatching membrane with the inner cuticular membrane attached; D, hatched (swimming) larva shortly after rupture of the hatching membrane; CS, cyst shell; ICM, inner cuticular membrane; 1, eye; 2, antenulla; 3, antenna; 4, mandible.

FIG. 2. The effects of NO donors Papa NONOate (A, B, G), Spermine NONOate (C, D) and Sin-1 Chloride (E, F) on the development of Bolshoye Yarovoe ARC-nmr.:1706 cysts was examined in capped tubes containing 95.0±0.5 mg cysts after an incubation time of 24 h (A, C, E, G) or 48 h (B, D, F, G). The extent of development is reported as the percent of developed cysts. A-F: Black bars, hatched larvae; grey bars, hatched and emerged larvae combined. Results are presented as mean values plus SE and the experiments were done in triplicate. G: Hatching percentage (H %) and hatching percentage plus (H⁺%) of Maloye Yarovoye cysts after 24 or 48 h treatment with 0.5, 1, 4 and 10 μM PapaNONOate or 6 ppm H₂O₂

FIG. 3. Promotion of Artemia cyst development by NO is inhibited by PTIO. Capped plastic tubes containing 95.0±0.5 mg of cysts in Instant Ocean® artificial sea water supplemented individually with 0.5 μM Papa NONOate, 6.0 μM Spermine NONOate, and 6.0 μM Sin-1 Chloride, were incubated with rotation for 48 hr in the presence (grey bars) and absence (black bars) of 300 μM PTIO. Control cysts were incubated in the absence of NO donors and PTIO (white bar). Results are presented as the mean values of three replicates with error bars representing SE. Letters indicate statistically significant differences (ANOVA and multiple comparisons, p<0.001).

FIG. 4. Hatching percentage (H %) of Maloye Yarovoye cysts after 24 or 48 h treatment with 0.5, 1, 4 and 10 μM PapaNONOate or 6 ppm H₂O₂.

FIG. 5. NO promotes adult development in diapaused pupae of Heliothis virescens (Lepidoptera: Noctuidae). Diapaused pupae were incubated in capped plastic Falcon tubes containing water supplemented with Spermine NONOate at concentrations of 10, 100 and 1000 μM. The time (days) needed to reach adult hatching was determined and the results are presented as the means±SD based on 20 pupae per treatment. Treatments were compared to controls with a Student's t-test.

FIG. 6. Termination of adult diapause in adults of L. decemlineata (being in diapause for 10 days) by NO gas at different concentrations (2.6, 6.1 and 67.4 ppmv). Data are expressed as the percentage of emerging adult beetles at different days after treatment with NO gas. (n=20).

FIG. 7. Termination of adult diapause in adults of L. decemlineata (being in diapause for 27 days) by NO gas at 0.300 ppmv. Data are expressed as the percentage of emerging adult beetles at different days after treatment with NO gas. (n=14).

### DETAILED DESCRIPTION

The present invention relates to methods for terminating dormancy in invertebrates wherein said dormant invertebrates are brought in contact with nitric oxide and/or a nitric oxide donor.

With the term dormancy is meant “a period of stasis in an organism's life cycle enabling the organism for example to deal with unfavorable changes in its environment”. During this period, growth, development, physical activity and reproduction can be temporarily and reversible suspended or suppressed. This definition includes quiescence and diapause. In one aspect dormancy is directly induced by adverse environmental conditions such as, but not limited to photoperiod, temperature, food, moisture, salt, oxygen chemicals or endocrine factors of other species. When said period is a seasonally recurring period, dormancy can also be called vernal dormancy (spring), aestivation (summer), autumnal dormancy (fall) or hibernation (winter).

Dormancy, more in particular diapause, may also occur during a genetically determined stage of metamorphosis, and its full expression develops in a species-specific manner, usually in response to a number of environmental stimuli that precede unfavorable conditions. Once dormancy has begun, metabolic activity is suppressed even if conditions favorable for development prevail.

Activity levels of dormant stages can vary considerably among species. Dormancy can occur at the embryonic (eggs, cysts), or at the post-embryonic stage, e.g. larval, pupal, or at the adult stage, depending on the species. Dormancy may occur in a completely immobile stage, such as the pupae and eggs, or it may occur in very active stages that undergo extensive migrations, such as the adult Monarch butterfly, Danaus plexippus. In cases where the invertebrate (insect) remains active, feeding is reduced and reproductive development is slowed or halted. In some species, dormancy is facultative and occurs only when induced by environmental conditions; in other species the dormancy period has become an obligatory part of the life cycle.

Hence in the method of the invention the dormant invertebrate can be in an embryonic or post embryonic stage.

With the term “metamorphosis” is meant, “a biological process by which an animal physically develops after birth or hatching, involving a conspicuous and relatively abrupt change in the animal's form or structure through cell growth and differentiation”. Some insects, amphibians, mollusks, crustaceans, cnidarians, echinoderms and tunicates undergo metamorphosis, which is usually (but not always) accompanied by a change of habitat or behaviour.

Dormancy can be found in aquatic (freshwater and marine) and terrestrial invertebrates. Thus, in the methods of the invention the dormant invertebrate can be an aquatic or a terrestrial invertebrate. Terrestrial invertebrates are invertebrates that live predominantly or entirely on land, as compared with aquatic invertebrates, which live predominantly or entirely in the water. Many invertebrates that are considered terrestrial have a life-cycle that is partly dependent on being in water. Many insects, for example, have an aquatic life cycle stage: their eggs need to be laid in and to hatch in water. After hatching there is an early aquatic form, either a nymph or larva. As used herein, the aquatic invertebrates are meant to include these known aquatic life cycle stages of terrestrial invertebrates.

In a particular embodiment of the present invention, the dormant invertebrate consists of an aquatic invertebrate, more in particular animal live feeds used in aquaculture for commercial larval rearing (hatcheries) of many species of fish and Crustacea, such as Artemia spp. (Crustacea), Daphnia spp. (Crustacea) and Brachionus spp. (Rotifera). In an even further embodiment the dormant invertebrate consists of any invertebrate but, i.e. except for the species used as animal live feed in aquaculture, in particular any invertebrate but, i.e. except for Artemia spp. In an alternative embodiment of the present invention, the dormant invertebrate consists of any aquatic invertebrate, including the species used as animal live feed in aquaculture, except for Artemia spp. In particular said dormant invertebrate can be any one from the Insecta, Crustaceae, Nematoda or Rotifera; more in particular any one from Crustaceae, Nematoda or Rotifera. Alternatively said dormant invertebrate consists of any terrestrial invertebrate; in particular Insecta.

Examples of freshwater and/or terrestrial phyla demonstrating dormancy include but are not limited to:
Dinophyta, Bacillariophyta and Ciliophora: planktonic protists,
Porifera: sponges,
Cnidaria: jellyfish, hydra, sea anemones and corals,
Aschelminthes: Gastrotricha,
   Rotifera: planktonic metazoan,
   Nematoda: roundworms or nematodes,
   Nematomorpha: horsehair or gordian worms,
Plathelminthes: flatworms,
Mollusca: bivalves, snails and cephalopodes,
Annelida: earthworms, polychaete worms and leeches,
Arthropoda: Crustaceans,
   subclass: Cephalocarida,
      Branchiopoda,
      Anostraca (Artemia spp)
      Ostracoda,
      Mystacocarida,
      Copepoda,
      Branchiura,
      Cirripedia,
      Malacostraca
   Insecta,
      subclass: Apterygota
         Pterygota
   Chelicerates,
      subclass: Merostomata
         Arachnida
         Pycnogonida
Tardigrada: water bears, lesser protostomes.
Lophosphora: Bryozoa,

Examples of exclusively or almost entirely marine phyla (many with planktonic larval stage) demonstrating dormancy includes but are not limited to:
Ctenophora: (comb-jellies) diverse planktonic phylum,
Chaetognatha: planktonic and predators,
Lophosphora: Phoronida: small phylum of tube-dwelling worms with lophosphore,
Pogonophora: small phylum of deep-water tube dwelling worms, lesser protostomes
Vestimentifera: small phylum of tube-dwelling worms associated with deep-sea seeps and vents, considered by some as Pogonophora,
Echinodermata: (starfish, urchins) large, diverse phylum,
Entoprocta: sometimes placed under Bryozoans,
Hemichordata: small phylum of burrowing worms,
Sipuncula: small phylum of burrowing worms,
Priapula: small phylum of burrowing worms,
Gnathostomulida: small phylum of tiny interstitial individuals,
Kinorhyncha: small phylum of tiny interstitial individuals,
Loricifera: small phylum of tiny interstitial individuals.

Examples of dormancy stages include but are not limited to:
resting eggs
(e.g. Order Cladocera, Anostraca, Notostraca and Conchostraca, Subclass Branchiopoda, Class Crustacea, Phylum Arthropoda),
diapausing eggs
(e.g. Order Coleoptera (beetles), Ephemeroptera (mayflies) and Trichoptera (caddisflies), Class Insecta, Phylum Arthropoda),
protected eggs or encysted eggs (cysts)
(e.g. resistant gelatinous egg mass Order Trichoptera (caddisflies), Class Insecta, Phylum Arthropoda),
gemmules also called reduction bodies
(e.g. in a family of freshwater sponges Family spongillidae, Phylum porifera; and in Phylum Bryozoa),
resistant cysts enclosing juvenile forms, adults or fragments of animals (e.g. Class Turbellaria, Phylum Plathelminthes; Subclass Oligochaeta, Phylum Annelida),
dehydrated individuals (anhydrobiose)
(e.g. small mucus lined cells in (Sub)class Hirudinea (leeches), Phylum Annelida),
immatures near the groundwater table
(e.g. (Sub)class Malacostraca (aquatic sow bugs, scuds, sideswimmers, crayfish, freshwater shrimp, opossum shrimp), Class Crustacea, Phylum Arthropoda),
juvenils in burrows
(e.g. Order Decapoda (crayfish, freshwater shrimp), (Sub)class Malacostraca, Class Crustacea, Phylum Arthropoda),
terrestrial pupae
(e.g. Order Trichoptera (caddisflies), Class Insecta, Phylum arthropoda),
resistant pupae
(e.g. Family Culicidae, Order Diptera (true flies), Class Insecta, Phylum arthropoda),
resting nymphs
(e.g. Order Odonata (dragonflies and damselflies) Class Insecta, Phylum arthropoda),
dauer larvae
(e.g. Order Rhabditida, (Sub)class secernenta, Class nematoda, Phylum Aschelminthes,
diapausing early instars
(e.g. Order Plecoptera (stoneflies) Class Insecta, Phylum arthropoda),
resistant late instar larvae sometimes in cocoons of silk or mucus
(e.g; Family Chironomidae, Order Diptera (true flies) Class Insecta, Phylum arthropoda),
burrowing adults
(e.g. Order Coleoptera (beetles), Class Insecta, Phylum arthropoda),
adults forming a protective epiphragm of dried mucus across the shell opening
(e.g. Class Gastropoda, Phylum Mollusca),
adults resistant to desiccation for a short time
(e.g. Subclass Ostracoda, Class Crustacea, Phylum Arthropoda), and
adults in cysts
(e.g. late copepodites Subclass Copepoda, Class Crustacea, Phylum Arthropoda)
reproductive dormancy
(e.g. order Diptera, (sub)class Pterygota).

As described previously environmental factors are important for terminating dormancy. Hence a person skilled in the art, when performing the methods of the invention must take into account the influence of external conditions. These conditions can be predetermined and/or adapted to the results and the effects of the method. Thus in one aspect of the present invention the methods are performed under predetermined conditions. Said predetermined conditions are one or more environmental conditions selected from the group of photoperiod, temperature, food, moisture, salt, oxygen, chemicals or endocrine factors from other subjects.

Examples of environmental factors regulating dormancy include but are not limited to:
short days maintaining and long days terminating dormancy in Nemobius yezoensis, Wyeomyia smithii and Meleoma signoretti,
short days maintaining dormancy and photoperiod having no active role in terminating dormancy in Chrysopa harrisii,
decreasing daylengths decelerating dormancy development, photoperiod sensitivity ceasing, and photoperiod having no active role in terminating dormancy, in the sweetclover weevil, Sitona cylindricollis; the European corn borer, Ostrinia nubilalis; the odonates, Tetragoneura cynosure and Lestes eurinus; the culicid, Chaoborus americanus; Pyrrhocoris apterus; the flesh flies, Sarcophaga bullata; the scarab, Anomala cuprea; Chrysopa carnea and Chrysopa harrisii,
short days maintaining and increasing daylengths terminating dormancy in Chrysopa downesi,
photoperiodic regimes during the predormancy larval stages influencing postdormancy reproduction in the tortricid Grapholitha funebrana.
cool conditions inducing and maintaining dormancy in the tropical flesh fly, Sarcophaga spilogaster,
the optimum temperature for dormancy development being higher than for nondormancy development and growth in the summer-diapausing mite Bdellodes lapidaria.
the optimum temperature for dormancy development being lower than generally occurring summer temperatures in the summer-diapausing lepidopterans, Mamastra brassicae and Leuhdorfia japonica,
hibernal dormancy ending more quickly under warm than under cold conditions in the cricket, Acheta commodus; the flesh flies, Sarcophaga argyrostoma and Sarcophaga bullata; the Colorado potato beetle, Leptinotarsa decemlineata; Chrysopa carnea; the southwestern corn borer, Diatraea grandiosella and the mosquito, Aedes atropalpus. In these species, temperature appears to play either no role or only a very small role in maintaining dormancy, and photoperiod is probably the primary dormancy maintaining factor, at least during late summer and early autumn; Subsequently, low temperatures may slow the rate of dormancy maintained by prevailing temperature conditions,
rising of the lower temperature threshold for dormancy development during the course of dormancy and short daylengths serving to maintain dormancy until spring in the gypsy moth, porthetria dispar, the flies, Pegomya betae and Chortophila brassicae, the fall webworm, Hyphantria cunea; the European red mite, Panonychus ulmi; the geometrid, Chesias legatella; the damselflies Lestes disjunctus and Lestis unguiculatus.
chilling terminating dormancy in Hyalophora cecropia,
temperatures during dormancy, as well as the duration of dormancy influencing postdormancy temperature reactions in Pleolophus basizonus and developmental cycles in several melolids,
temperature conditions during dormancy affecting the rate of postdormancy development in Laspeyresia nigricans, Neodirprion sertifer and Panonychus ulmi and influencing the rate of post dormancy growth, as well as the number of postdormancy instars, in the dragonfly, Aeshna mixta.
Post dormancy reproductive potential being related to temperature conditions during dormancy in Neodiprion sertifer,
postdormancy development being prevented, sometimes for a considerable period, until temperatures rise above the thermal threshold for development in Sitona cylindricollis; the lady beetle, Coccinella septempunctata; Pyrrhocoris apterus; the flesh fly Sarcophaga bullata; the thrips, Anaphothrips obscures; Chrysopa carnea and Chrysopa harrisii,
optimal temperature being required for normal growth and development after dormancy in the twostriped grasshopper, Melanoplus bivittatus; Ostrinia nubilalis; several dragonfly species; Neodiprion sertifer; the ichneumonid, Pleolophus basizonus; the winter moth, Operophtera brumata; Hyphantria cunea; the mite, Halotydeus destructor; the arctiid, Spilosoma lubricipeda; the leafhopper, Nephotettix cincticeps; the mayfly, Ephemerella ignite; the larch sawfly, Pristiphora erichisonii; the moth, Exoteleia nepheos; the forest tent caterpillar, Malacosoma disstria; and Chrysopa carnea,
food-induced summer dormancy being ended by food alone in Chrysopa carnea mohave strain in the laboratory,
dormancy being maintained in the laboratory until prey is available in Chrysopa carnea mohave strain after the photoperiodically sensitive phase of a photoperiodically induced autumnal dormancy is completed,
the diet received during dormancy influencing the duration of adult dormancy in Chrysopa carnea mohave strain,
food having a role in terminating the larval hibernal dormancy in Chaoborus americanus, the quality of the diet during dormancy not influencing dormancy duration,
water intake being required to complete dormancy, after photoperiodic maintenance of dormancy ends, in the European corn bore, Ostrinia nubilalis,
water being required for initiating postdormancy embryogenesis in the mosquito Aedes vexan,
contact moisture required after dormancy in order to hatch in several species of damselflies in the genus Lestes,
water required for postdormancy development but not for dormancy development in larvae of Cephus cinctus and Diatraea grandiosella, and
moisture increasing the rate of postdormancy development in Pectinophora gossypiella,
a hormonal stimulus from the geometrid host Bupalus piniarius being required, in order to end dormancy in the tachinid parasite, Eucarcelia rutilla,
premature dormancy termination in Calliphora vicina being correlated with parasitism by the braconid, Apaereta minuta; this may result from the direct effect of the parasite's hormonal system on the host larval tissues, or it may result from the influence of the parasite on the host hormonal system,
allochemicals regulating settlement (a metamorphosis like process in marine invertebrate taxa) such as a coral effluent in the coral-eating nudibranch Phestilla sibogae, a riboflavin degradation product in the solitary ascidian Halocynthia roretzi, a peptide released by conspecific adults in the sand dollar Dendraster excentricus, and coralline algae as in the coral Acropora millepora.

Dormancy, growth and metamorphosis in invertebrates are controlled by hormones synthesized by endocrine glands near the front of the body. The neuroendocrine system of insects consists primarily of neurosecretory cells in the brain, the corpora cardiaca, corpora allata and the prothoracic glands. There are several key hormones involved in the regulation of dormancy, metamorphosis and growth: juvenile hormone (JH), dormancy hormone (DH), and prothoracicotropic hormone. Note that the convention thus far has been to refer to these nonvertebrate hormones as “thyroid hormones” based on chemical similarity, despite the fact that, with the possible exception of tunicates,

Examples of neuroendocriene factors regulating dormancy, metamorphosis, development and growth include but are not limited to:
ecdysteroids maintaining dormancy, and inhibiting further development in the gypsy moth. In this moth, dormancy intercedes at a later stage of development, after embryonic development has been completed but before the first instar larva breaks to the chorion,
ecdysteroids and juvenile hormone (JH), regulating all the manifold and profound morphological changes that occur between the worm-like larva and the winged adult in holometabolous beetles, bees, butterflies, and flies,
clusters of neurons on the protocerebrum called the pars lateralis maintaining reproductive dormancy by inhibiting JH production by the corpora allata in the bean bug, Riptortus pedestris,
JH being required for the accumulation by the fat body of a storage protein that is associated with dormancy in the corn borer, Diatraea gradiosella.
JH titer remaining elevated in some Lepidoptera species, thus guaranteeing that any molt will be stationary rather than progressive.
Dormancy hormone (DH) regulating embryonic dormancy in the eggs of the silkworm moth, Bombyx mori. DH is released from the subesophogeal ganglion of the mother and triggers trehalase production by the ovaries. This generates high levels of glycogen in the eggs, which is converted into the polyhydric alcohols glycerol and sorbitol. Sorbitol directly inhibits the development of the embryos. Glycerol and sorbitol are reconverted into glycogen at the termination of dormancy,
a factor from the mesothorax repressing the action of a development-promoting factor from the abdomen, in the giant silkmoth Antheracea yamamai, but thus far, the identity of neither factor is known,
thyroid hormones (THs) regulating morphological transformation during metamorphosis in 3 classes spanning 12 families: Echinoidea (sea urchins, sea biscuits, and sand dollars), Asteroidea (sea stars) and Ophiuroidea (brittle stars) and the metamorphic transition from benthic polyp to pelagic jellyfish in scyphozoans (Cnidaria), and
an unidentified “head hormone” and a JH-like metamorphic hormone regulating the metamorphic-like process in annelids.

A number of indices can be utilized to test the termination of dormancy in invertebrates. In the methods of the present invention indices to test termination of dormancy can be selected from one or more of a change in an embryonic stage, a post embryonic stage, an endocrine factor, an expression of a gene, an expression of a protein, a signaling process, the state of the reproductive system, an enzyme activity, a metabolic activity, the composition of the cuticle, the respiration rate, and the behavior more in particular the feeding behavior.

Such indices include but are not limited to:
alterations in response to environmental factors such as photoperiod, temperature, allochemicals, food, moisture, oxygen and/or salt,
changes in life stage,
changes in neuroendocrine factors such as juvenile hormone (JH), dormancy hormone (DH), prothoracicotropic hormone and other thyroid hormones as described above,
change in the state of the reproductive system,
metabolic changes such as changes in fat reserves, glycogens and polyols,
changes in number of layers of hydrocarbons for waterproofing the cuticle,
change in respiration rate and changes in genes linked to oxygen consumption,
changes in feeding behaviour such as for example an indication of dormancy termination in species that undergo dormancy as adults or as free-living immatures and that have observable changes in their response to food during dormancy. The accuracy of behavioural changes as indices of dormancy termination in nature depends on how closely timed they are to reactivation of the endocrine system,
changes in signalling processes including growth factors, cell-death machinery and possibly efflux transport
changes in the insulin-like signalling pathway
change in expression of several clock genes including period, timeless (ls-tim,s-tim) dClock, cycle, doubletime and vrille, as well as several forms of cryptochrome, genes that encode a photoreceptor involved in circadian rhythmicity,
changes in genes and proteins associated with dormancy such as proteins related to E26 transforming sequence proteins, early dormancy genes (e.g. pScD41) late dormancy genes (e.g. usp) and genes expressed intermittently during dormancy (e.g. po),
changes in genes and proteins associated with the synthesis and cellular action of ecdysteroids such as ecr coding for the ecdysone receptor (EcR), usp coding for ultraspiracle (USP) the dimerization partner of EcR, chr75a and chr75b coding for the ecdysone-inducible transcription factors CHR75A and CHR75B, and chr3 coding for hormone receptor 3.
changes in genes and peptides associated with the Drosophila gene capability, capa).
changes in cell cycle regulators such as cyclin dependent kinases, cyclin E, p21, p53 and proliferating cell nuclear antigen (pcna),
changes in genes that regulate G2/M transition
changes in stress proteins and stress responsive genes such as heat shock proteins HSP23, HSP70, HSP90, and dauer regulatory protein; molecular chaperones such as p26; cold-induced genes such as Samui; members of the immunoglobulin family such as sarcotoxins and hemolin; salt-stress proteins such as an Arabidopsis gene AtNOA1 encoding an NO-associated protein,
changes in stress related pathways such as that of cytochrome oxidases, efflux pumps, transporters
changes in enzymes such as alkaline phosphatases,
changes in midgut enzymes such as trypsin, chymotrypsin, elastase, aminopeptidase and esterases,
enzymatic changes that take place for example in preparation for cold hardening, in production of glycogens, in conversion of glycogens to polyols or transferring phosphate to the mitochondria,
changes in the presence of storage proteins that are present in the hemolymph and remain there in abundance throughout dormancy. When dormancy is terminated they quickly disappear from the hemolymph. These hemolymph proteins are also referred to as dormancy-associated proteins. These hexameric proteins can have a high content of aromatic amino acids and are then classified as arylphorins,

With the term “NO-donor” is meant a synthetic or natural chemical compound that release NO. This term also encompass NO-donors. This release can be continuously over a period of time and under physiological conditions. Among the most widely used NO donors are organic nitrates (for e.g., glycerin trinitrate) and nitrites, and furoxan derivatives. These donors require thiols as a cofactor for generating NO and can use endogenous sources of thiols. NO is first transferred to thiol and it is then released from the S-nitrosothiol.

A number of NO donors have been developed to augment the action of intracellularly released NO, which are also known to stimulate guanylate cyclase activity. Some of the known natural carriers of NO are S-nitrosoglutathione, S-nitrosocysteine and sodium nitroprusside (SNP). Upon conversion of thiols to S-nitrosothiols, NO is released by a homolytic break of the S—N bond (2 RS—NO→RS—SR+2.NO). The bioaction of S-nitrosothiols is reported to be similar to that of NO in most cases. However, the use of S-nitrosothiols has a disadvantage in that they are too unstable to be used as long term NO donors. S-Nitroso-N-penicillamine (SNAP) offers somewhat higher stability when compared to other nitroso compounds. Although the release of NO is spontaneous, it is somewhat sustained. Glyco-SNAPs (e.g. Glyco-SNAP-1 or Glyco-SNAP-2) are more stable analogs of SNAP and the release of NO can be monitored over a period of 24 to 30 hours.

A versatile group of stabilized NO-amine complexes known as NOC compounds has also been developed. These donors release NO spontaneously without the influence of any cofactors. They act as intramolecular zwitterions, stabilized with an intramolecular hydrogen bond through dispersion of the negative charge, which prevents protonation. In these compounds the rate of NO release is, therefore, dependent upon the weakness of the hydrogen bond. These compounds, when dissolved in aqueous medium, such as buffer, plasma, or cell culture medium, dissociate to form two NO molecules and one molecule of the corresponding amine. The half-life of NO release varies from a few minutes to several hours. Examples of NOC compounds are Diethylamine NONOate (a.k.a. Diethylammonium (Z)-1-(N,N-diethylamino)diazen-1-ium-1,2-diolate; DEA NONOate; DEA/NO; having CAS N^{o} 56329-27-2), Diethylamine NONOate/AM (a.k.a. ((3,3-Diethyl-2-oxidotriazanyl)oxy)methyl acetate), Spermine NONOate (a.k.a. (Z)-1-[N-[3-aminopropyl]-N-[4-(3-aminopropylammonio)butyl]-amino]diazen-1-ium-1,2-diolate; having CAS N^{o} 136587-13-8), Papa NONOnate (a.k.a. (Z)-14N-(3-aminopropyl)-N-(n-propyl)amino]diazen-1-ium-1,2-diolate; Propylamine Propylamine NONOate; having CAS N^{o} 146672-58-4), Sin-1 Chloride (a.k.a 5-amino-3-(4-morpholinyl)-1,2,3-oxadiazolium chloride; Linsidomine; 3-Morpholino-sydnonimine; having CAS N^{o} 16142-27-1), NOC-5 (a.k.a. 3-(Aminopropyl)-1-hydroxy-3-isopropyl-2-oxo-1-triazene; having CAS N^{o} 146724-82-5), NOC-7 (a.k.a. 3-(2-Hydroxy-1-methyl-2-nitrosohydrazino)-N-methyl-1-propanamine; having CAS N^{o} 146724-84-7), NOC-9 (a.k.a. 6-(2-Hydroxy-1-methyl-2-nitrosohydrazino)-N-methyl-1-hexanamine; MAHMA NONOate; having CAS N^{o} 146724-86-9), NOC-12 (a.k.a. 1-Hydroxy-2-oxo-3-(N-ethyl-2-aminoethyl)-3-ethyl-1-triazene; having CAS N^{o} 146724-89-2), or NOC-18 (a.k.a. N-[bis(2-aminoethyl)amino]-N-hydroxynitrous amide; having CAS N^{o} 146724-94-9).

Still another series of NO donors known as the NOR compounds (e.g. NOR-1 (a.k.a. (+/−)-(E)-[(E)-hydroxyimino]-6-methoxy-4-methyl-5-; (+/−)-(E)-[(E)-hydroxyimino]-6-methoxy-4-methyl-5-; nitro-3-hexenamide; nitro-3-hexenamide; having CAS N^{o} 163032-70-0) or NOR-3 (a.k.a. (+/−)-(E)-Ethyl-2-[(E)-hydroxyimino]-5-nitro-3-hexeneamide; having CAS N^{o} 163180-49-2) has been developed. These non-thiol-based compounds release NO spontaneously, under physiological conditions, in a rate-controlling manner and their by-products do not exhibit any significant biological activity. The pattern of NO release is very similar to that of NOC series of compounds. The half-life of NOR compounds varies from a few minutes to a few hours. NOR compounds are relatively stable in anhydrous organic solvents.

Thus in the method of the invention the NO-donor can be selected from Spermine NONOate, Papa NONOnate, Sin-1 Chloride, S-nitrosoglutathione, S-nitrosocysteine, sodium nitroprusside, S-Nitroso-N-penicillamine (SNAP), Glyco-SNAP-1, Glyco-SNAP-2, Diethylamine NONOate, Diethylamine NONOate/AM, NOC-5, NOC-7, NOC-9, NOC-12, NOC-18, NOR-1, or NOR-3. In a particular embodiment the NO-donor is selected from the group consisting of PapaNONOate, Spermine NONOate, and Sin-1 Chloride.

As will be apparent for the skilled artisan and evident from the examples hereinafter, the optimal final concentration of the NO-donor as used in the methods of the present invention, will be dependent on the NO-donor used and on the dormant invertebrate treated. In the below examples, the aqueous medium/solution was supplemented with one or more NO-donors at a final concentrations of at least 0.05 μM; in particular at least 0.1 μM; in particular at least 0.5 μM; in particular between 10 μM and 10 mM; even more in particular between 100 μM and 1 mM; more in particular between 0.5 μM and 60 μM.

As evident from the examples hereinafter, for aquatic invertebrates (e.g. animal live feeds used in aquaculture for commercial larval rearing (hatcheries) of many species of fish and Crustacea) the NO-donors are applied in the medium, e.g. artificial seawater, typically used to grow (culture) said aquatic invertebrate. As will be apparent for the skilled artisan, the optimal concentration will be dependent on the NO-donor used and on the dormant aquatic invertebrate treated. In the below examples, the aqueous medium was supplemented with one or more NO-donors at a final concentrations of at least 0.05 μM; in particular at least 0.1 μM; in particular at least 0.5 μM; in particular between 0.5 μM and 1 mM; more in particular between 0.5 μM and 60 μM, and the aquatic invertebrate was incubated with said supplemented medium from 10 h to 48 h under standard culturing conditions.

It is accordingly a further objective of the present invention to provide a method for terminating dormancy in an aquatic invertebrate characterized in that said dormant invertebrate is brought in contact with a nitric oxide donor. In particular by supplementing the medium, e.g. artificial seawater, typically used to grow (culture) said aquatic invertebrate with the NO-donors; more in particular with the NO-donors at final concentrations of at least 0.05 μM; in particular at least 0.1 μM; in particular at least 0.5 μM; even more in particular between and about 0.5 to 500 μM; alternatively at a final concentration between and about 0.5 to 100 μM; even more in particular at a final concentration of between and about 0.5 to 60 μM. The dormant aquatic invertebrate is typically incubated in said supplemented medium under standard culturing conditions, such as for example from 10 h to 72 h; in particular for 10, 12, 16, 18, 20, 24 h, 48 h, or 72 h.

As evident from the examples hereinafter, for terrestrial invertebrates the NO-donors are for example applied by contacting the dormant terrestrial invertebrate with an aqueous solution of the NO-donors. As will be apparent for the skilled artisan, the optimal concentration will be dependent on the NO-donor used and on the dormant terrestrial invertebrate treated. In the below examples, the dormant terrestrial invertebrates were contacted with an aqueous solution of the NO-donors at final concentrations of at least 0.05 μM; in particular at least 0.1 μM; in particular at least 0.5 μM; in particular between 10 μM and 10 mM; even more in particular between 100 μM and 1 mM; and the invertebrates being exposed to said aqueous solution for a period from about 15 min to 48 h, in particular from about 30 min to 2 h.

It is accordingly a further objective of the present invention to provide a method for terminating dormancy in a terrestrial invertebrate characterized in that said dormant terrestrial invertebrate is brought in contact with a nitric oxide donor. In particular by exposing said dormant terrestrial invertebrate to an aqueous solution of an NO-donor; more in particular with the NO-donor at a final concentration between and about 100 μM and 10 mM; alternatively at a final concentration between and about 500 μM and 5 mM; even more in particular at a final concentration of about 1 mM. The dormant terrestrial invertebrate is typically exposed to said aqueous solution for a period from about 15 min to 48 h, in particular from about 30 min to 2 h.

Also NO-based prodrugs, with protective groups attached, may be used to obtain a targeted release of NO. Furthermore, compounds that mimic the effect of NO can be used to obtain the same effect. Examples include but are not limited to cyanide, hydrogen peroxide and ethylene.

Instead of using NO or NO-donors, interference with NO-generating pathways is also possible, hence mimicking the effect of NO or a NO donor. For example upregulation of the expression or activity of NO-synthase gene and/or protein. The transcription machinery is considered to be one of the main targets for NO action, especially for genes involved in long-term adaptive responses. In this sense, the NO-mimicking effect can be accomplished through the modification of transcription factors, receptors or enzymes that then activate or inactivate a signalling cascade. Also mimicking a direct action of NO on a final target is possible, such as the ending of chain-propagated reactions or enzymatic activation by direct modification. For example, in animals, many NO functions are accomplished through stimulation of soluble guanylate cyclase (GC) and consequent increase in cGMP levels. In addition, calcium and calmodulin have been described as alternative components in NO-mediated signaling.

NO can also be delivered by bacteria such as denitrifying bacteria, methanotrophic bacteria and heterotrophic bacteria or through NO in gaseous form. Hence the present invention also encompass a method for terminating dormancy in an invertebrate wherein said dormant invertebrate is brought in contact with NO delivered by microorganisms, in particular bacteria, or with gaseous NO.

NO and NO donors can be used in combination with other compounds such as but not limited to cyanide and/or hydrogen peroxide.

The present invention is also concerned with the use of NO or NO donors as defined above for terminating dormancy or for studying termination of dormancy. The dormant invertebrate in the method of the invention can be unfavorable or beneficial. Termination of dormancy can be determined using any of the indices described above, in particular termination of dormancy can be determined using one or more of a change in an embryonic stage, a post embryonic stage, an endocrine factor, an expression of a gene, an expression of a protein, a signaling process, the state of the reproductive system, an enzyme activity, a metabolic activity, the composition of the cuticle, the respiration rate, and the behavior more in particular the feeding behavior NO and NO-donors can be used for pest management. NO and NO donors can be used to break dormancy of unfavourable species at timeperiods when the environmental conditions are bad, resulting in death of these species. Hence, NO and NO donors can be used to eliminate unfavourable invertebrates in pest control management. Decrease of the life cycle span can also have negative effects on reproduction.

Alternatively NO and NO-donors can be used to synchronise post dormancy development. As discussed above, except for species that require a specific stimulus to end dormancy, the occurrence of dormancy termination within a population often is distributed over a considerable time span, which makes it difficult to destroy unfavourable species with just one treatment. Hence, synchronisation of the dormancy termination followed by treatment of the unfavourable species with pesticides will be more effective and hence requires less pesticides and/or biocides.

Thus, the use of NO or NO donors as defined above for terminating dormancy can be followed by treatment with a control agent such as but not limited to chemicals, pesticides, insecticides, or biocides.

Examples of treatment of unfavourable species include but are not limited to:
breaking or preventing dormancy in the Colorado potato beetle Leptinotarsa decemlineata,
breaking of pupal dormancy of butterflies such as in the cotton budworm Heliothis virescens,
breaking of dormancy of dormant stages of Hemiptera (whiteflies, aphids) followed by treatment of all non-dormant stages with insecticides.
breaking of dormancy of dormant stages of Acari (spidermites) followed by treatment of all non-dormant stages with insecticides/acaricides.
breaking of dormancy in all hidden stages (eggs and nites) of lice's in plants and humans followed by treatment with biocides which will also have a direct effect on reproduction,
breaking and synchronization of cysts of plantpathogenic nematodes such as Globodera spp.

In beneficial species, synchronisation with for example crop development can be obtained. Examples include but are not limited to:
termination of hibernation and synchronisation in cultures of Bombus spp. such as Bombus terrestris to enhance pollination of tomato plants,
termination of dormancy and synchronisation of entomopathogenic insects and worms such as respectively Encarsia spp. (e.g. Encarsia formosa), Orius spp. and Heterorhabditis spp (e.g. Caenorhabditis elegans).

Furthermore in commercial important species such as for example the silkmoth, NO and NO donors can be used to terminate dormancy of the eggs and hatching resulting in a positive effect on silk production by Bombyx mori. Hence NO and NO donors can be used for terminating dormancy in the production of silk.

Animal live feeds are used in aquaculture for commercial larval rearing (hatcheries) of many species of fish and Crustacea. Several types of such animal live feeds exist such as Artemia spp. (Crustacea), Daphnia spp. (Crustacea) and Brachionus spp. (Rotifera). These species can be stored and transported as eggs and cysts. NO and NO-donors can be used to synchronize and accelerate dormancy termination. For Artemia freshly hatched naupliae (Instar I=first larval stage) have a higher nutritional value than 2 or 3 day old naupliae. Hence, NO and NO donors can be used, for example introduced in the hatching medium to produce live food, in particular for aquacultures. In a further or specific embodiment the present invention provides the use of NO and NO donors in a method to produce free swimming Artemia nauplii, starting from an amount of cysts.

The introduction of aquatic species in resting life stages by the release of ballast water is a less well-known but potentially important invasive species vector. Best-management practices designed to minimize transport of ballast water cannot eliminate this threat, because residual water and sediment are retained in ballast tanks after draining Chemical treatment can be used for treatment of residual material in ship ballast tanks, however reduced toxicity in the presence of sediment raises serious doubts as to the potential of chemical biocides to kill aquatic invertebrate resting stages buried in sediment retained in ship ballast tanks. By first treating the sediments and the ballast water with NO and NO donors and breaking dormancy of the aquatic invertebrates followed by treatment with chemicals can be an effective strategy to circumvent this problem. Hence NO and NO donors can be used to eliminate aquatic invertebrates in ballast water.

Phenological studies, particular those pertaining to dormancy maintenance, termination, and postdormancy development are vital to both theoretical and applied biology because environmental adaptions underlie the interactions of species with their physical and biological environments. Thus the uses of the invention must take into account the fluctuating abiotic factors and their influence on the species involved as well as the interactions between the changing biotic factors (e.g. the crop, the pest, and the beneficial species).

The agents and organisms described herein can be packaged as a kit. Thus, one or more agents or organisms can be present in a first container, and the kit can optionally include one or more agents or organisms in a second container. The kit can include instructions describing the method of the present invention. The agents, organisms, containers and/or the instructions can be present in a package. Thus the present invention encompasses a kit comprising NO, a NO donor or NO delivering microorganisms and dormant invertebrates.

The contents of the kit can contain but is not limited to gaseous NO, NO donor(s), NO delivering microorganisms, dormant invertebrates, medium, buffers, etc. It is also possible that NO, NO donor and/or NO delivering microorganisms are mixed with and/or adhered or coated onto said dormant invertebrate. Hence the invention also encompass a container comprising NO, a NO donor or NO delivering microorganisms and a dormant invertebrate wherein said NO, NO donor or NO delivering microorganisms are mixed with and/or adhered or coated onto said dormant invertebrate. Such as for example shown in the examples hereinafter, the amount of NO-donor added per gram cysts will be dependent on the NO-donor used and can easily be determined by the skilled artisan based on the desired final concentration in the medium, and is for example at least 4 μg NO-donor per g cysts.

### EXPERIMENTAL PART

### Example 1

### Effect of NO-Donors on Post-Diapause Development of Encysted Artemia Embryos

1a. Effect of NO-Donors on Post-Diapause Development of Bolshoye Yaravoye Strain Encysted Artemia Embryos

### Materials and Methods

### Artemia Cysts

Encysted embryos (cysts) from a parthenogenetic strain of Artemia obtained in winter 2005-2006 in Bolshoye Yarovoye (Altay Region: 52° 51′64″ N-78° 37′32″ E), Russia (Artemia Reference Center (ARC) code number BY 1706), were stored in the dark at 4° C. in dry sealed bags. For the experiment, the cysts were grown at 26-28° C. with constant illumination in 25 ml of sea water under varying experimental conditions as detailed below.

### Promotion of Cyst Development by NO

Tightly capped 50 ml plastic tubes containing 95.0±0.5 mg of Artemia cysts in artificial seawater (32 g salt/l, Instant Ocean®) supplemented separately with the NO donors (final concentrations between 0-60 μM)
3-(2-hydroxy-2-nitroso-1-propylhydrazino)-1-propanamine(PapaNONOate) (half life 15 min)
N-[4-[1-(3-aminopropyl)-2-hydroxy-2-nitrosohydrazinobutyl]-1,3-propane diamine (Spermine NONOate) (half life 39 min)
3-morpholine syndnonimine (Sin-1 Chloride) (half life 20 hr)
6 ppm H₂O₂ final solution
   were incubated with rotation (4 rpm) at 28° C.+/−1° C., under continuous illumination.

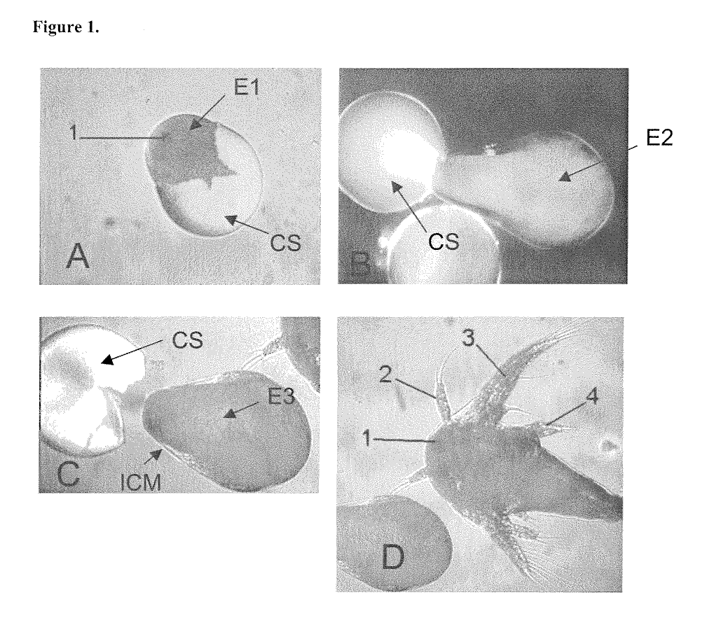

To quantify larval (nauplii) emergence and hatching (FIG. 1) which served as measures of cyst development, 6 samples of 250 μl from each 50 ml tube were mixed individually with 250 μl of sea water and 2 drops of Lugol's solution (Van Stappen, G, 1996) prior to counting nauplii (N) and umbrella (U) with the aid of a dissecting microscope. Two drops of NaOCl (14% (technical) active chlorine) and NaOH (32% w/v) were then added to dissolve cyst shells, revealing non-hatched (E) embryos for counting. The extent of development was determined as the percent of developed cysts which was calculated as either the number of hatched larvae, or the number of hatched and emerged larvae, obtained from 100 cysts, the latter represented by the sum of hatched nauplii (larvae), emerged larvae and undeveloped cysts containing embryos.

The hatching percentage (H %) was calculated according to:

$$H = \frac{N}{N + U + E} * 100 \%$$

The hatching percentage plus (H⁺%) was calculated according to:

$$H = \frac{N + U}{N + U + E} * 100 \%$$

To determine if development was dependent on NO generation, capped 50 ml plastic tubes containing 95.0±0.5 mg of cysts in artificial sea water (Instant Ocean®) supplemented individually with 0.5 μM Papa NONOate (MW: 176.2-conc: 88 μg/l-46.3 μg/g cysts), 6.0 μM Spermine NONOate (MW: 262.4-conc: 1.5 mg/l-0.79 mg/g cysts) or 6.0 μM Sin-1 Chloride (MW: 206.7-conc 1.2 mg/l-0.63 mg/g cysts) were incubated with rotation for 48 hr after addition of the NO scavenger 2-phenyl-4,4,5,5,-tetramethylimidazoline-1-oxyl 3-oxide (PTIO) (Sigma-Aldrich) to 300 μM. The percent of developed cysts was calculated as described above.

### Results

### NO Promotes the Post-Diapause Development of Artemia Cysts

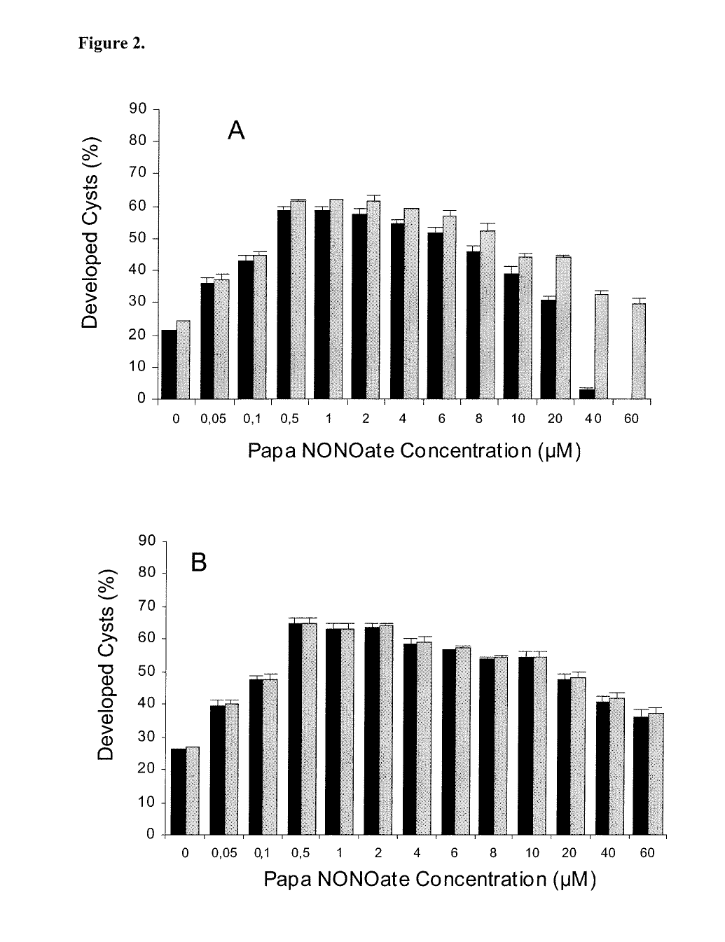

Incubation for 48 hr with any of the three NO donors used in this study yielded almost equal amounts of hatched nauplii (larvae) with few emerged nauplii (larvae) remaining. For all three NO donors tested, an increase in developed cysts was already observed at a concentration of 0.05 μM and 0.1 μM, however Papa NONOate was most effective at a lower concentration (0.63 μM) than either Spermine NONOate (4.0 μM) or Sin-1 Chloride (4.0 μM) (FIG. 2A-F). Maximum cyst development obtained with NO donors was approximately 64%. NO donors at concentrations of 40 or 60 μM, tended to reduce the overall extent of development achieved after 48 hr (FIGS. 2B, D and F), an effect most prominent with Papa NONOate. Moreover, after 24 hours, Papa NONOate inhibited hatching almost completely at 40 and 60 μM, an effect overcome by 48 hr (FIGS. 2 A and B), whereas the effects of Spermine NONOate or Sin-1 Chloride at these concentrations were less dramatic, but obvious (FIG. 2C-F). The hatching percentage (H %) and hatching percentage plus (H⁺%) after 24 or 48 h of treatment with 0, 0.5, 1 and 4 mM Papa NONOate or 6 ppm H₂O₂ are shown in FIG. 2G.

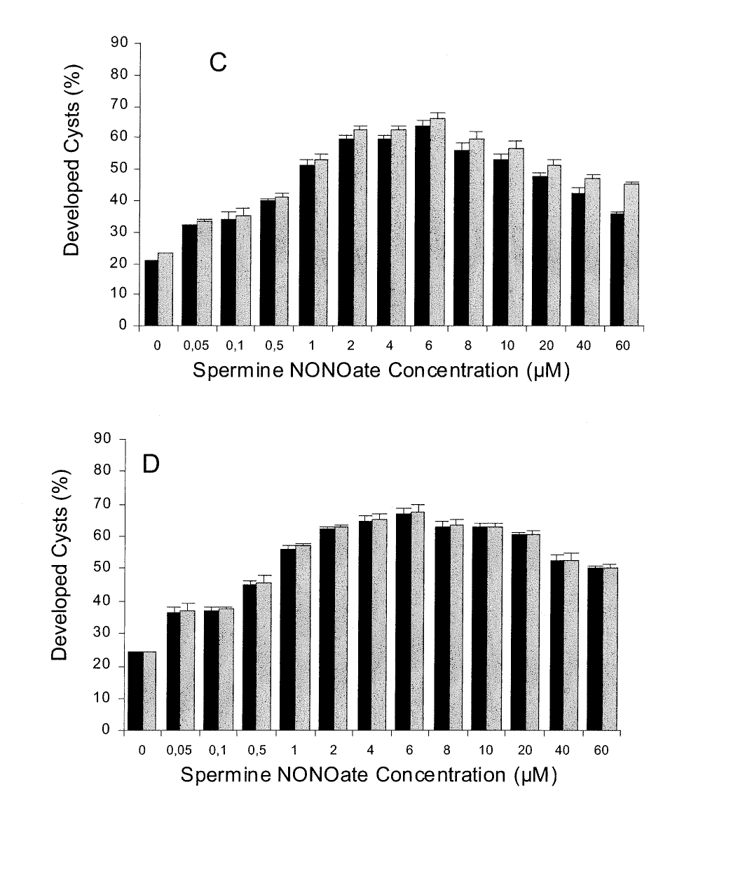

Addition of 300 μM PTIO to tubes containing sea water supplemented individually with 0.5 μM Papa NONOate, 6.0 μM Spermine NONOate and 6.0 μM Sin-1 chloride inhibited cyst development (FIG. 3), indicating that cyst development is dependent on NO generation.

1b. Effect of NO-Donors on Post-Diapause Development of Maloye Yarovoye Strain encysted Artemia Embryos

### Materials and Methods

### Artemia Cysts

Encysted embryos (cysts) from a parthenogenetic strain of Artemia obtained in 2008 in Maloye Yarovoye (Altay Region: 53° 04′00″ N-79° 10′00″ E), Russia (Artemia Reference Center (ARC) code number MY 1732), were stored in the dark at 4° C. in dry sealed bags. For the experiment, the cysts were grown at 26-28° C. with constant illumination in 25 ml of sea water under varying experimental conditions as detailed below.

### Promotion of Cyst Development by NO

Tightly capped 50 ml plastic tubes containing 50.0±0.5 mg of Artemia cysts in 25 ml artificial seawater (32 g salt/l, Instant Ocean®) supplemented separately with
0.5, 1, 4 or 10 μM of the NO donor PapaNONOate (3-(2-hydroxy-2-nitroso-1-propylhydrazino)-1-propanamine) (half life 15 min)
6 ppm H₂O₂ final solution
   were incubated with rotation (4 rpm) at 28° C.+/−1° C. under continuous illumination.

To quantify nauplii (larvae) emergence and hatching (FIG. 1) which served as measures of cyst development, 6 samples of 250 μl were taken from each 50 ml tube after 24 and 48 h. Subsequently the obtained samples were mixed individually with 250 μl of sea water and 2 drops of Lugol's solution (Van Stappen, G, 1996) prior to counting nauplii (N) and umbrella (U) with the aid of a stereomicroscope. Two drops of NaOCl (14% (technical) active chlorine) and NaOH (32% w/v) were then added to dissolve cyst shells, revealing non-hatched (E) embryos for counting.

The hatching percentage (H %) was calculated according to:

$$H = \frac{N}{N + U + E} * 100 \%$$

The hatching percentage plus (H⁺%) was calculated according to:

$$H = \frac{N + U}{N + U + E} * 100 \%$$

### Results

### NO Promotes the Post-Diapause Development of Artemia Cysts

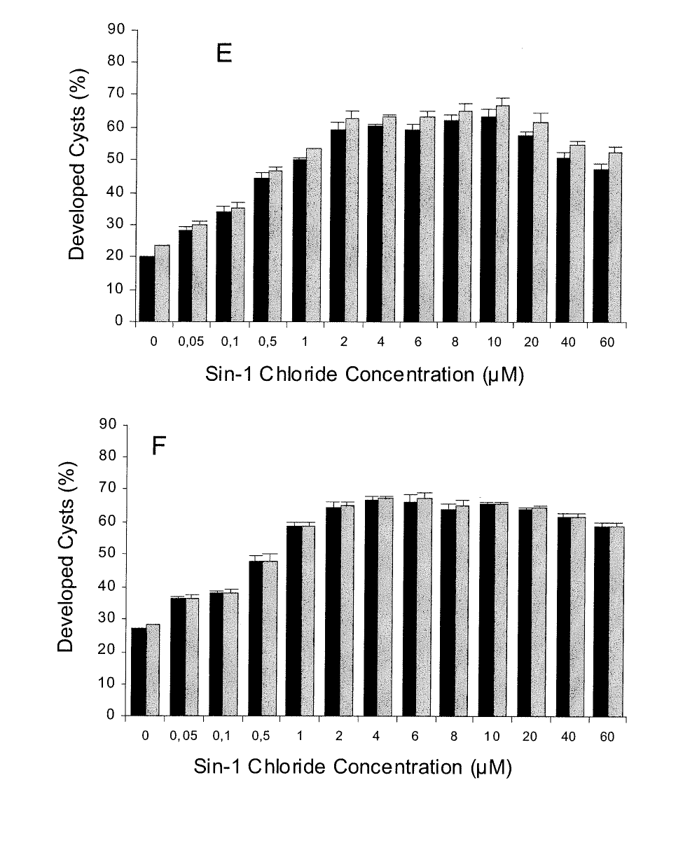

The hatching percentage (H %) and hatching percentage plus (H⁺%) after 24 or 48 h of treatment with 0, 0.5, 1 and 4 μM Papa NONOate or 6 ppm H₂O₂ are shown in FIG. 4. The control treated cysts showed a hatching percentage of around 10% after 48 h, whereas treatment with 4 μM PapaNONOate, showed a significantly higher hatching percentage of 47%. So, PapaNONOate increased the number of nauplii almost 5-fold, compared to the control. Furthermore, the optimal doses of PapaNONOate for inducing the hatching of Maloye Yarovoye in diapausing cysts is 4 μM.

1c. Effect of NO-Donors on Post-Diapause Development of Vinh Chau Strain Encysted Artemia Embryos

### Materials and Methods

### Artemia Cysts

Dry and processed embryos (cysts) from a strain of Artemia obtained in 2009 in the Soc Trang Province in the Mekong Delta (09° 19′48″ N-105° 58′11″ E) (Artemia Reference Center (ARC) code number VC 1742), were stored in the dark at 4° C. in tin cans, that were not resealed after the first opening. For the experiment, the cysts were grown at 26-28° C. with constant illumination in 25 ml of sea water under varying experimental conditions as detailed below.

### Promotion of Cyst Development by NO

Tightly capped 50 ml plastic tubes containing 50.0±0.5 mg of Artemia cysts in 25 ml artificial seawater (32 g salt/l, Instant Ocean®) supplemented with 0.5, 1, or 4 μM of the NO donor PapaNONOate (3-(2-hydroxy-2-nitroso-1-propylhydrazino)-1-propanamine) (half life 15 min) were incubated with rotation (4 rpm) at 28° C.+/−1° C. under continuous illumination.

To quantify nauplii (larvae) emergence and hatching (FIG. 1) which served as measures of cyst development, 6 samples of 250 μl were taken from each 50 ml tube after 10, 12, 14, 16, 18, 20, 22 and 24 h post incubation. Subsequently the obtained samples were mixed individually with 250 μl of sea water and 2 drops of Lugol's solution (Van Stappen, G, 1996) prior to counting nauplii (N) and umbrella (U) with the aid of a stereomicroscope. Two drops of NaOCl (14% (technical) active chlorine) and NaOH (32% w/v) were then added to dissolve cyst shells, revealing non-hatched embryos (E) for counting.

The hatching percentage (H %) was calculated according to:

$$H = \frac{N}{N + U + E} * 100 \%$$

The hatching percentage plus (H⁺%) was calculated according to:

$$H = \frac{N + U}{N + U + E} * 100 \%$$

### Results

### NO Promotes the Post-Diapause Development of Artemia Cysts

As evident from table 1, the hatching percentages of Vinh Chau cysts after treatment with 1 and 4 μM PapaNONOate for 16 hours and more, are comparable to the non-treated controls. However, for the NO treatment with 0.5 μM, the hatching percentage, is significantly increased after 16 and 18 h post-treatment. Furthermore, although the effect is not significant, for all treatments, the cysts already start to hatch at 10 or 12 hours post-treatment (indicated in grey), whereas for the untreated control, first hatching is only observed after 14 hours post-treatment.

TABLE 1
Hatching percentage (H%) of Vinh Chau cysts that underwent NO-
treatments of 0.5, 1 and 4 μM.

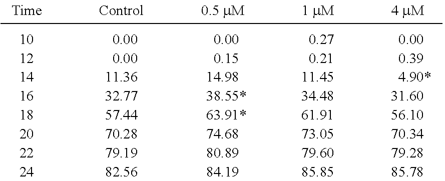

*indicate significant difference compared to untreated control, at the 0.05 level, using a 95% confidence interval

As shown in table 2, for the hatching percentages plus (H⁺%), the effects of treatment with PapaNONOate are even more pronounced compared to the hatching percentages, with a significantly increase in H⁺% for all treatments at 12, 18 and 20 hours compared to non-treated controls (indicated in grey). Furthermore, at 10, 16 and 24 hours there is a significant increase for at least one treatment (i.e. 0.5 μM; 0.5 μM+4 μM; and 1 μM respectively), with a trend to increased levels for the other treatments at the same time points.

TABLE 2
Hatching percentage plus (H⁺%) of Vinh Chau cysts that underwent NO-
treatments of 0.5, 1 and 4 μM.

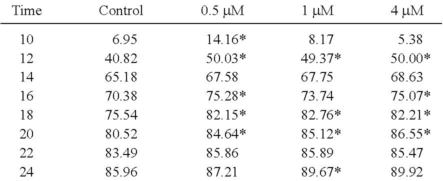

*indicate significant difference compared to untreated control, at the 0.05 level, using a 95% confidence interval

In conclusion, although the final hatching after 24 hours is comparable between treated and untreated Vinh Chau cysts, treatment with PapaNONOate, clearly increased the hatching rate for the Vinh Chau cysts during the initial period of the hatching process (10-22 h). The optimal dose was determined to be 0.5 μM PapaNONOate.

### Example 2

### Effect of SpermineNONOate in Diapaused Pupae of Heliothis virescens

### Material and Methods

The larval stages of the tobacco budworm Heliothis virescens (Lepidoptera: Noctuidae) were reared on an artificial diet at standard conditions of 25° C. and long light conditions with 14 h light (Smagghe G et al., 1994). To produce nondiapause pupae, the larvae were kept at these conditions throughout development, whereas for pupal diapause induction, third instar larvae were transferred to 18° C. and short light conditions of 8 h light. The pupal stemma were examined to check the diapause status as described (Philips J R et al., 1966). Over 85% of the pupae entered diapause under those conditions.

Diapaused pupae of H. virescens were dipped for 30 minutes in a water solution of the NO-donor SpermineNONOate (purchased from Sigma) at 10, 100 and 1000 μM. The treatment was done in capped plastic Falcon tubes of 10 ml. Controls were dipped in distilled water. After treatment, pupae were put in 9-cm Petri dishes, lined with a filter paper and kept at 25° C. and 14 h light, and development of adults was scored at daily intervals. Per treatment, 20 pupae were tested. Means±SD were compared to controls with a Student's t-test.

### Results

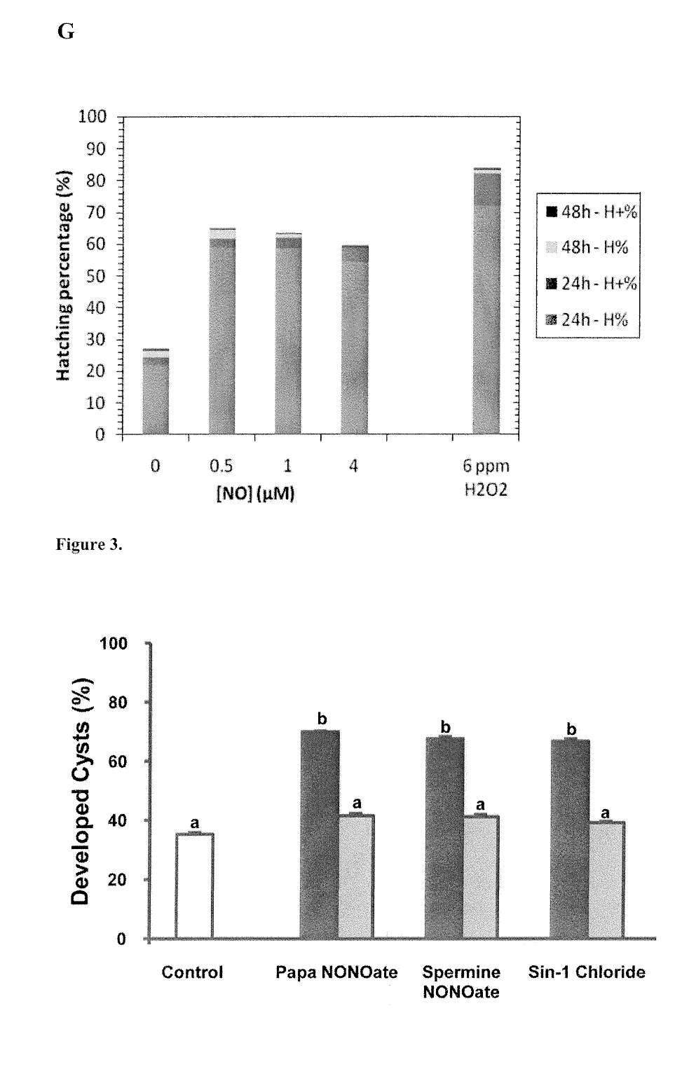

The pupae that had been dipped in 1000 μM, developed into the adult stage after 7.60±0.89 days, which was significantly (p=0.01) faster than in the control (9.17±0.75 d). The two lower concentrations scored similar (p≧0.05) as in the controls (FIG. 5).

### Example 3

### Effect of Sin-1 Chloride on Branchionus Resting Egg Hatching

### Material and Methods

Resting eggs of Brachionus were suspended in 20 ml seawater (25 g/l), supplemented with different concentrations of Sin-1 Chloride (1-60 μM) and put in a falcon tube. These tubes were put on a rotor at 28° C. under constant illumination. The effect on the hatching rate was measured after 48 hr and 72 hr of incubation, by counting emerged rotifers in function of the amount of NO donor added.

### Results

The results illustrate that in the presence or in the absence of the NO donor Sin-1 Chloride, ultimately between 35 and 40% of the resting eggs seem to hatch (table 3). Yet in the presence of 10 μM Sin-1 Chloride the hatching is faster, as after 48 hr incubation, 30% of the eggs had hatched (while in the negative control only 12.8% had hatched). After 72 h, the hatching in the negative control and in the treatment with 10 μM Sin-Chloride is not different. Note that in comparison with hatching in Artemia cysts (example 1), higher concentrations of NO seem to slow down the hatching of Branchionus resting eggs.

TABLE 3
Percentage hatching of resting eggs of Branchionus after 48 hr or 72 hr
incubation with varying concentrations of Sin-1 Chloride

| | 48 hrs | 72 hrs |
|---|---|---|
| Untreated | 12.84% (1.36) | 34.95% (2.21) |
| 1 μM Sin-1 Chloride | 12.34% (3.62) | 33.11% (2.66) |
| 6 μM Sin-1 Chloride | 18.93% (1.25) | 33.71% (0.66) |
| 10 μM Sin-1 Chloride | 30.04% (3.10) | 37.17% (2.88) |
| 20 μM Sin-1 Chloride | 17.99% (3.18) | 32.38% (2.63) |
| 60 μM Sin-1 Chloride | 14.98% (3.52) | 27.57% (1.00) |

### Example 4

### Diapause Termination of Dormancy and Development in Eggs with Diapaused Embryos of Bombyx mori

### Material and Methods

All experiments were carried out on silkmoth strain Bombyx mori J106 (Lepidoptera: Bombycidae). Larvae were maintained on an artificial diet (Yakuruto Co.) under standard conditions of 25° C., 65% relevant humidity and 16 h light (Tazima Y, 1978; Swevers et al., 2003). Egg batches were collected from a continuous insect colony and used for the experiments.

Eggs with diapaused embryos of B. mori were dipped for 30 minutes in a water solution of the NO-donor SpermineNONOate (purchased from Sigma) at 1 mM. The treatment was done in capped plastic Falcon tubes of 10 ml. In parallel, eggs with diapaused embryos were dipped for 5 minutes in 20% HCl (in water) at 48° C., and then the eggs were washed with water and dried to the air. The latter treatment is the normally used procedure to break diapause in eggs with diapaused embryos for maintenance of the insect colony of B. mori (Tazima Y, 1978; Swevers et al., 2003). In a water-control, eggs with diapaused embryos were dipped in distilled water only, and in a blank-control, eggs with diapaused were not treated. After treatment, eggs were put in 9-cm Petri dishes, lined with a filter paper and kept at 25° C. at 16 h light, and development of neonate larvae (=hatching of first instar larvae from eggs) was scored at daily intervals. The numbers of eggs with diapaused embryos per treatment (n) are given with the results.

### Results

As shown in Table 4, 86% (60/70) of the eggs with diapaused embryos that had been dipped in 1 mM SpermineNONOate, developed into neonate larvae at 6-8 days after treatment. At day 6, already 40% of the eggs (28/70) had developed into neonates (first instar larvae). Another 31% (22/70) and 14% (10/70) of eggs with diapaused embryos developed into neonates at day 7 and day 8 respectively. In total only 14% (10/70) of the eggs with diapaused embryos did not develop into neonate larvae within 14 days.

TABLE 4
NO terminates embryonic diapause in B. mori with development into
neonate larvae.

| | | | | | | Number |
|---|---|---|---|---|---|---|
| | | | | | | and |
| | | | | | | percent- |
| | | | | | | age of un- |
| | | Number and percentage of | | | | developed |
| | | developed neonates at day | | | | eggs at |
| Treatment | n | 6 | 7 | 8 | 14 | day 14 |
| SpermineNONOate | 70 | 28 | 22 | 10 | 0 | 10 |
| | | (40%) | (31%) | (14%) | (0%) | (14%) |
| 20% HCl | 72 | 10 | 36 | 12 | 0 | 14 |
| | | (14%) | (50%) | (17%) | (0%) | (19%) |
| Water-control | 66 | 0 | 0 | 0 | 0 | 66 |
| | | (0%) | (0%) | (0%) | (0%) | (100%) |
| Blank-control | 62 | 0 | 0 | 0 | 0 | 62 |
| | | (0%) | (0%) | (0%) | (0%) | (100%) |

Treatment of the eggs with 20% HCl for 5 minutes at 48° C., which is normally used to terminate embryonic diapause for the insect culture maintenance of B. mori, resulted in development into neonate larvae of 81% (58/72) of the eggs within 6-8 days. More in details, 14% (10/72) of the eggs had developed into neonates at day 6, 50% (36/72) at day 7 and 17% (12/72) at day 8; 19% (14/72) of the eggs did not develop within 14 days after treatment. As expected, there was no development of the eggs with diapaused embryos into neonate larvae in the water-control and the blank-control within 14 days after treatment.

### Conclusion

In conclusion, the treatment of eggs with diapaused embryos with SpermineNONOate at 1 mM in water can terminate the embryonic diapause in B. mori, and this procedure was as efficient as is normally done with 20% HCl in water to maintain an insect colony of B. mori.

### Example 5

### Diapause Termination of Dormancy and Development in Eggs with Diapaused Embryos of the Silkmoth Bombyx mori by NO Gas

### Material and Methods

All experiments were carried out on silkmoth strain Bombyx mori J106 (Lepidoptera: Bombycidae). Larvae were maintained on an artificial diet (Yakuruto co.) under standard conditions of 25° C., 65% relative humidity and 16 h light (Tazima, Y., 1978; Swevers et al., 2003). Egg batches were collected from a continuous insect colony and used for the experiments.

In a first series, a batch of 70 eggs with diapaused embryos of B. mori were submerged for 5 minutes in 20% HCl (in distilled water) at 48° C., and then the eggs were washed with distilled water and dried to the air. The treatment was done in capped plastic Falcon tubes of 10 mL. The latter treatment is the normally used procedure to break diapause in eggs with diapaused embryos for maintenance of the insect colony of B. mori (Tazima, Y., 1978; Swevers et al., 2003). In a water-control, a batch of 70 eggs with diapaused embryos was submerged in distilled water for 5 minutes at 45° C., and in a blank-control, the same procedure was followed, but without addition of water.

In parallel, 4 batches of 70 eggs each, were put in Pyrex glass bottles of 100 mL, which were sealed gastight by means of a rubber stop and a screw cap. Then the air was removed from these bottles by inserting a needle (in the rubber stop) connected to a vacuum pump. Using 50 ppmv (Parts Per Million by Volume) NO gas (with N2 as carrier gas, purchased from Air Liquide) dilution series were prepared (with air as dilutor) to obtain final NO concentrations of 20, 200, 2000 and 20000 ppbv, in these bottles. Verification of the NO concentrations was done by sampling from a parallel bottle that received the same gas mixture as the treatment bottle and NO content was determined by using a chemiluminescence detector (CLD 77 Amsp, Eco Physics). 30 minutes after insertion of the gas mixtures, the rubber stops were removed. These treatments were all performed at room temperature.

After all the treatments, the eggs were put in separate 9-cm Petri dishes, lined with a filter paper and kept at 25° C., 65% relative humidity and 16 h light. Development of the neonate larvae (=hatching of first instar larvae from eggs) was scored at daily intervals. The numbers of eggs with diapaused embryos per treatment (n) are given with the results.

### Results

Treatment of eggs containing diapauzed embryos of B. mori with NO gas ranging between 0.02-20 ppmv resulted in termination of diapauze (Table 5). At 5 days after treatment 27.1-37.1% of the eggs had emerged in the treatments with NO gas. In contrast, in the controls that did not receive NO gas, none (0%) of the eggs with diapaused embryos developed into neonate larvae.

TABLE 5
Termination of embryo diapause in eggs of B. mori by NO gas at different
concentrations (0.02-20 ppmv). Data are expressed as percentages of
hatching of first instar larvae from eggs, and this at 5 days after treatment.

| [NO] ppmv | Percentage of developing eggs |
|---|---|
| blank (no NO gas) | 0 |
| 0.02 | 37.1 |
| 0.20 | 35.7 |
| 2.00 | 31.4 |
| 20.0 | 27.1 |

### Conclusions

In conclusion, the treatment of eggs with diapaused embryos with NO-gas can terminate the embryonic diapause in B. mori, and this procedure was at least as efficient as is normally done with 20% HCl in water to maintain an insect colony of B. mori.

### Example 6

### Diapause Termination of Dormancy and Development in Diapaused Adults of the Colorado Potato Beetle Leptinotarsa decemlineata by NO Gas

### Material and Methods

All experiments were carried out on the Colorado potato beetle (potato beetle), Leptinotarsa decemlineata (Say) (Coleoptera: Chrysomelidae). Diapaused adult beetles were obtained by rearing larvae and pupae at 8 h light, 16 h dark, 25±2° C. and 70% relative humidity (Yi, et al., 2000). All life stages were fed potato (Solanum tuberosum L. ‘Bintje’) plants, that were refreshed daily. 15 days after adult emergence, the diapausing adults were placed in individual aluminum foil, 2.5-cm diameter ‘beakers’, containing fresh soil (Universal potting soil, Type 1, M. Snebbout n.v., Kaprijke, Belgium), and were stored at 4° C. in darkness.

Under red light, beetles were collected and put in Pyrex glass bottles of 100 mL, containing a layer of fresh soil. These bottles were wrapped in a double layer of aluminum foil to ensure complete darkness and sealed gastight by means of a rubber stop and a screw cap. The air was then removed by inserting a needle (in the rubber stop) connected to a vacuum pump. Using 50 ppmv NO gas (with N₂ as carrier gas, purchased from Air Liquide), dilution series were prepared (with air as dilutor), and the desired final NO concentrations were injected in each bottle. A blank control, received ambient air only. Verification of the NO concentrations was done by sampling from a parallel bottle that received the same gas mixture as the treatment bottle. NO content was determined with a chemiluminescence detector (CLD 77 Amsp, Eco Physics). The treatments were all done at 4° C. in the dark and 30 minutes after insertion of the gas mixtures, the rubber stops were removed.

After the treatments, the beetles were collected in plastic containers (dimensions: 10×21×5 cm), containing fresh soil and potato leaves and kept at 25±3° C., 65% relative humidity and 8 h light (Exp. 1 and 2). In the last experiment (Exp 3), beetles were left at 4° C. in the dark for 6 hours before transfer to a 12° C. incubator, in the dark. The beetles that emerged were collected every day and the beetles' emergence per treatment (% of total) are given with the results.

### Results

### Exp 1.

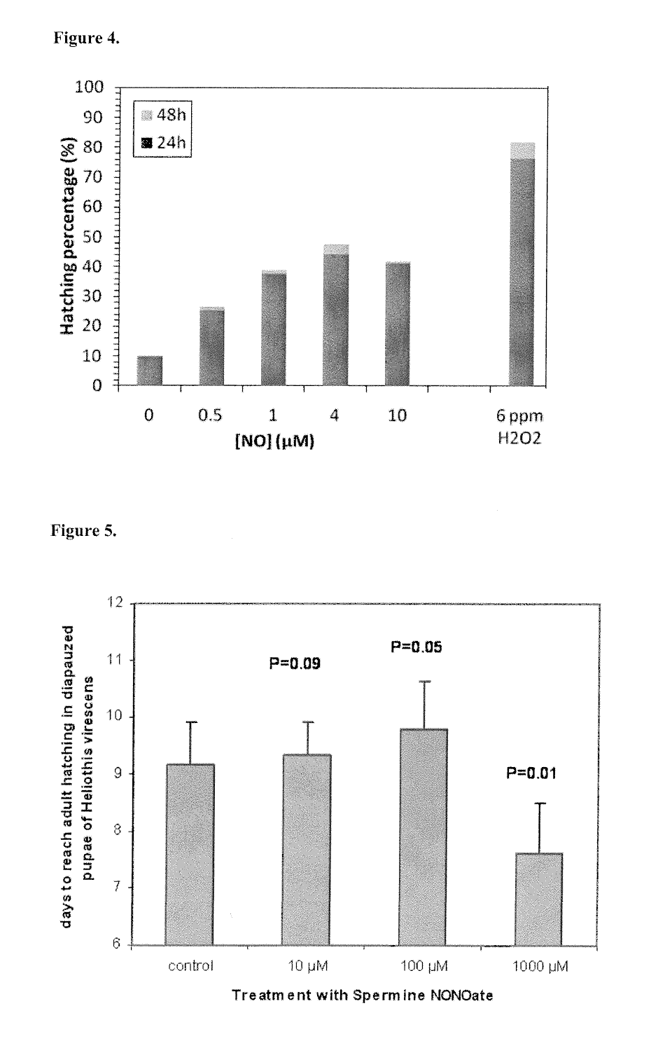

As shown in FIG. 6, treatment with 2.6 and 6.1 ppmv NO gas caused a termination of the adult diapause in 20% of the beetle adults (that had been in diapause for 10 days) and this happened at 3 days after the treatment. These adults emerged as normal active (non-diapaused) adults from the soil. At day 6, the percentage had increased to 40% for 2.6 ppmv NO gas, while that for 6.1 ppmv remained 20%. For the treatment with the highest dose, i.e. 67.4 ppmv NO, 20% of the diapaused adults had emerged after 4 days.

In contrast, in the blank control samples, that received no NO gas, no adults emerged even not at 7 days after the treatment.

### Exp. 2.

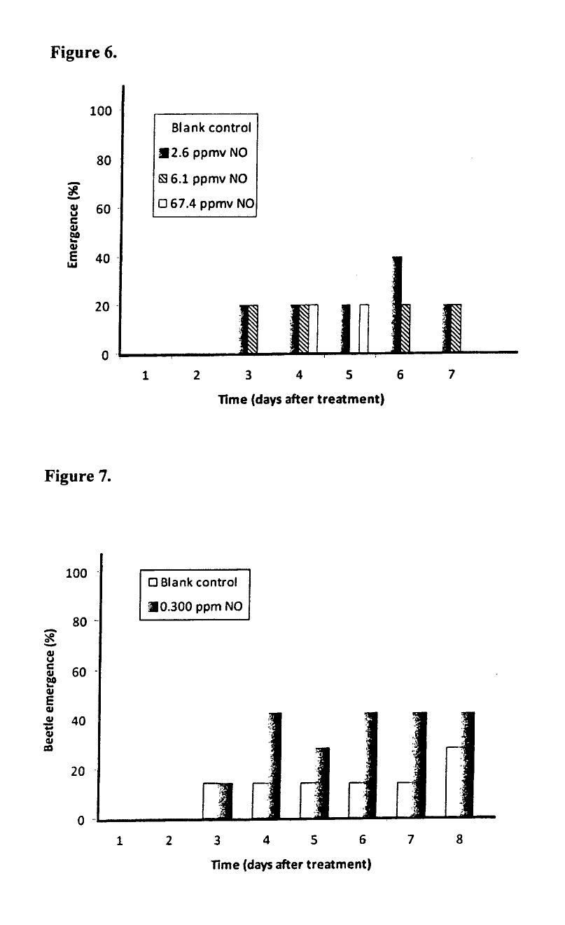

With adults of L. decemlineata, that were in diapause for 27 days, 42.9% of the adult beetles that had been treated with 0.300 ppmv NO gas, had emerged from the soil after 4 days (FIG. 7). In the blank controls, that received no NO gas, 14.3% of the beetles had emerged after 4 days, and only after 8 days, this percentage had increased to 28.6%.

### Conclusions

In conclusion, the treatment of diapausing individuals with NO-gas (tested at 0.3, 2.6 and 6.1 ppmv) can terminate the diapause in adult L. decemlineata. In contrast, control animals that received no NO-gas treatment merged not (with use of adults being in diapause for 10 days), or later and to a lower extend (with use of adults being in diapause for 27 days) compared to the NO-gas treated ones.

### REFERENCES

Bishop C D, Brandhorst B P, 2003, Nitric oxide signaling metamorphosis, and the evolution of biphasic life cycles, Evol. & Dev, 5: 542-50.
Bishop, C D, Huggett M, Heyland A, Hodin J, Brandhorst B P, 2006, Interspecific variation in metamorphic competence in marine invertebrates: the significance for comparative investigations of regulatory systems. Integr Comp Biol, 46: 662-682.
Champlin D T, Truman J W, 2000, Ecdysteroid coordinates optic lobe neurogenesis via a nitric oxide signaling pathway, Development, 127: 3543-3551.
Gammie S C, Truman J W, 1999, Eclosion hormone provides a link between ecdysis-triggering hormone and crustacean cardioactive peptide in the neuroendocrine cascade that controls ecdysis behavior, J. Exp. Biol., 202: 343-352.
Hodin J, 2006, Expanding networks: Signaling components in and a hypothesis for the evolution of metamorphosis, Integregrative and Compar. Biology, 46: 719-742.
Philips J R, Newsom L D, 1966, Diapause in Heliothis zea and Heliothis virescens (Lepidoptera: Noctuidae), Ann Entomol Soc Am, 59: 154-159.
Röszer T, Czimmerer Z, Szentmiklosi A J, Banfalvi G, 2004, Nitric oxide synthesis is blocked in the central nervous system during dormant periods of the snail Helix lucorum, Cell and Tissue Research, 316: 255-262.
Smagghe G, Degheele D, 1994, Action of a novel nonsteroidal ecdysteroid mimic, tebufenozide (RH-5992), on insects of different orders, Pestic Sci, 42: 85-92.
Swevers L, Kravariti L, Ciolfi S, Xenou-Kokoletsi M, Ragoussis N, Smagghe G, Nakagawa Y, Mazomenos B, and Iatrou K, 2003, A cell-based high-throughput screening system for detecting ecdysteroid agonists and antagonists in plant extracts and libraries of synthetic compounds, FASEB J. 17: (14), 134-136.
Tazima Y, 1978, The silkworm: An Important Laboratory Tool, Kodansha Books
Teodoro R O, O'Farrell P H, 2003, Nitric oxide-induced suspended animation promotes survival during hypoxia, EMBO Journal, 22: 580-587.
Van Stappen G., 1996, Artemia: introduction, biology and ecology of Artemia, Manual on the production and use of live food for aquaculture FAO Fisheries Technical Paper, 361: 295 pp.
Yi, S.-X. and Adams, T. S., 2000, Effect of pyriproxyfen and photoperiod on free amino acid concentrations and proteins in the hemolymph of the Colorado potato beetle, Leptinotarsa decemlineata (Say), Journal of Insect Physiology 46: (10) 1341-1353

1. A method for terminating dormancy in an invertebrate comprising contacting said dormant invertebrate with at least one of nitric oxide or a nitric oxide donor.

2. The method of claim 1 wherein the dormant invertebrate is in an embryonic or a post-embryonic stage.

3. The method as claimed in claim 1 wherein the dormant invertebrate is an aquatic or terrestrial invertebrate.

4. The method as claimed in claim 1, wherein the dormant invertebrate is an aquatic invertebrate.

5. The method as claimed in claim 1 wherein the dormant invertebrate is a terrestrial invertebrate.

6. The method as claimed in claim 1 wherein the dormant invertebrate is selected from the Insecta, Crustaceae, Nematoda or Rotifera species.

7. The method as claimed in claim 1 wherein the method is performed under predetermined environmental conditions selected from the group of photoperiod, temperature, food, moisture, salt, oxygen, chemicals and endocrine factors.

8. (canceled)

9. The method as claimed in claim 1 wherein said intervertebrate is contacted with a nitric oxide donor selected from Spermine NONOate, Papa NONOnate, Sin-1 Chloride, S-nitrosoglutathione, S-nitrosocysteine, sodium nitroprusside, S-Nitroso-N-penicillamine (SNAP), Glyco-SNAP-1, Glyco-SNAP-2, Diethylamine NONOate, Diethylamine NONOate/AM, NOC-5, NOC-7, NOC-9, NOC-12, NOC-18, NOR-1, or NOR-3.

10. The method as claimed in claim 4, wherein the dormant invertebrate is contacted with a nitric oxide donor dissolved in an aqueous medium supplemented with one or more nitric oxide donors.

11. The method according to claim 10, wherein the medium is supplemented with said nitric oxide donors at a final concentration of at least 0.05 mM.

12. The method according to claim 11, wherein the dormant invertebrate is contacted with the nitric oxide donor for 10 to 72 hours.

13. The method as claimed in claim 1 wherein the nitric oxide is delivered by microorganisms.

14. The method as claimed in claim 1, wherein termination of dormancy is determined using one or more of a change in an embryonic stage, a post embryonic stage, an endocrine factor, an expression of a gene, an expression of a protein, a signalling process, the state of the reproductive system, an enzyme activity, a metabolic activity, the composition of the cuticle, the respiration rate, or the feeding behavior.

15. A kit comprising nitric oxide, a nitric oxide donor or nitric oxide delivering microorganisms, and a dormant invertebrate.

16. A container comprising nitric oxide, a nitric oxide donor or nitric oxide delivering microorganisms, and a dormant invertebrate; wherein said nitric oxide, nitric oxide donor or nitric oxide delivering microorganisms are mixed with and/or adhered or coated onto said dormant invertebrate.

17. (canceled)

18. (canceled)

19. The method as claimed in claim 1 including producing live aquacultures.

20. The method as claimed in claim 1 for the treatment of pests.

21. The method as claimed in claim 20 further including treatment with a pest control agent.

22. (canceled)

23. (canceled)
